(19) United States
(12) Patent Application Publication
(10) Pub. No.: US 2005/0260737 A1
(43) Pub. Date: Nov. 24, 2005

(54) Novel lipase gene from Bacillus sphaericus 205y.

(76) Inventors: Raja Noor Zaliha Abd. Rahman, Selangor (MY); Abu Bakar Salleh, Selangor (MY); Mahiran Basri, Selangor (MY); Chin John Hun, Selangor (MY)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/084,508

(22) Filed: Mar. 18, 2005

(30) Foreign Application Priority Data
Mar. 18, 2004 (MY) 20040958

(51) Int. Cl. C12N 1/21; C12Q 1/04

(52) U.S. Cl. 435/252.31; 435/34

(57) ABSTRACT

A biologically pure culture of Bacillus sphaericus strain 205y capable of producing an organic solvent-tolerant lipase was isolated from soil samples via direct plating method using 1% (v/v) of either benzene, toluene, or mixture of benzene and toluene as their sole carbon source, ethyl benzene and ρ-xylene. The lipase gene was isolated via genomic library and sequenced. The production of lipase gene from Bacillus sphaericus strain 205y is a novel gene. The lipase gene was cloned from Bacillus sphaericus strain 205y and the recombinant lipase was efficiently excreted into the culture medium using expression vectors bearing the lipase gene.

### FIELD OF INVENTION

The present invention relates to the fields of Molecular Biology and Enzyme technology. More particularly this invention relates to a newly isolated organic solvent-tolerant Bacillus sphaericus 205y and a novel lipase from Bacillus sphaericus 205y

### BACKGROUND OF THE INVENTION

Lipases or triacylglycerol hydrolases E.C. 3.1.1.3 are enzymes that hydrolyse ester bonds of triglycerides at the oil-water interface. Lipases (E.C. 3.1.1.3) belong to the carboxylic ester hydrolase family. Lipases have the ability to hydrolyse long-chain acylglycerols (≧C₁₀) whereas esterases hydrolyse ester substrate with short-chain fatty acids (≦C₁₀). Lipases are widely distributed in plants, animals and microorganisms and have a broad range of properties with respect to positional specificity, fatty acid specificity, thermostability, pH optimum, etc (Philips et. al. 1995). Most of the lipases used in biotechnology industries were originated from microorganisms.

The versatility of lipases especially those from microbial lipases have made them a good candidate for industrial exploitation. Uses of substrates of the lipase are often insoluble or partially soluble in water and utilizing organic solvents are in favour for some reactions. Using of organic solvents also provide many advantages: 1) the relative high solubility of substrate, 2) the relative ease recovery of products in organic phase, 3) the possibility of reducing the degree of undesirable substrate and/or product inhibition in organic solvent-water two-phase systems and 4) the ability to shift the reaction equilibrium in the synthetic direction by continuously removing the products in the organic solvent-water two phase systems. Organic solvents are generally known to have detrimental effect on microorganisms and the enzyme they produced. In the presence of organic solvents, most organisms lose their functions and cease growing. Enzymes are often denatured and inactivated by organic solvents (Ogino, et al. 1994).

### SUMMARY OF THE INVENTION

The invention discloses a new organic solvent-tolerant Bacillus sphaericus 205y that is capable of growing in the presence of BTEX and cloning of a novel gene from Bacillus sphaericus 205y.

### DESCRIPTION OF THE INVENTION

The invention will be described in detail with referral to a preferred embodiment and to the drawings in which:

### BRIEF DESCRIPTION OF THE FIGURES

FIG. 1: Effect of different production medium on lipase activity. Assay was conducted at 24 h ( [symbol] ) and 48 h (□) incubation time in triplicate. Bars corresponded to standard deviation.

FIG. 2: Recombinant plasmid of 205y pLIP containing 9 kb insert. Lane 1: Recombinant plasmid 205y pLIP digeseted with Bam HI releasing the insert and vector; Lane 2: undigested recombinant plasmid of clone 205y pLIP; Lane 3: Lambda DNA/Hind III (bp) and Lane 4: pUC 19 digested with Bam HI.

FIG. 3: Restriction Endonuclease Map of 205y pLIP. The coloured bar represents the chromosomal DNA corresponding to lipase gene derived from Bacillus sphaericus 205y. Uncoloured box represent part of the plasmid pUC 19. Restriction enzymes used are: E, Eco RI; S, Sac I; H, Hind III; X, Xba I.

FIG. 4: Subcloning of the DNA coding Bacillus sphaericus 205y lipase from 205y pLIP. The lipolytic phenotype was determined by halo formation on tributyrin-amp agar. Coloured box represent the foreign insert. Uncoloured box represent part of the pUC 19.

FIG. 5: Nucleotide sequence of the lipase gene from B. sphaericus 205y (SEQ ID NO:4). The putative −35, −10 promoters, Shine-Dalgarno ribosome binding site, start and stop codon and the stem loop inverted repeat termination sequences were underlined. The amino acid sequence shown in FIG. 5 is SEQ ID NO:5.

FIG. 6: Similarity between lipase from B. sphaericus 205y (AF 453713) (SEQ ID NO:5) and B. subtilis (M74010) (SEQ ID NO:17) and B. pumilis (A34992) (SEQ ID NO:18). Numbers in bracket are the accession numbers of the bacteria. Boxed amino acid showed the catalytic motif A-X-S-X-G (SEQ ID NO:16). Abbreviation use are B.sub: B. subtilis, B.pum: B. pumilis, B.sph: B. sphaericus 205y.

FIG. 7: Alignment of amino acid sequences from ten lipases of various organisms. The abbreviation used were 205y: Bacillus sphaericus 205y (AF 453713) (SEQ ID NO:5), Xy.sp: Xyxella sp. (XF1253) (SEQ ID NO:19), S.so: Sulfolobus solfataricus (AAK42652) (SEQ ID NO:20), P.sp.: Psychrobacter sp (AAF70342) (SEQ ID NO:21), M.sp: Moraxella sp. (P24484) (SEQ ID NO:22), B11-1: Pseudomonas sp B11-1 (AAC38151) (SEQ ID NO:23), B.ha: Bacillus halodurans (BAB05967) (SEQ ID NO:24), D.ra: Deinococcus radiodurans (AAF10396) (SEQ ID NO:25), LipH: M.tuberculosis (LipH) (Z95586) (SEQ ID NO:26), and LipQ: M.tuberculosis (LipQ) (D70868) (SEQ ID NO:27). The boxed sequenced are the conserved pentapeptide (Gly-X-Ser-X-Gly) (SEQ ID NO:8) region and His-Gly dipeptide region.

FIG. 8: Phylogenetic analysis of lipase gene from Bacillus sphaericus 205y.mDendogram showing sequence relationships between Bacillus sphaericus 205y (AF 453713) and Xyxella sp. (XF1253), Sulfolobus solfataricus (AAK42652), Psychrobacter sp (AAF70342), Moraxella sp. (P24484),Pseudomonas sp B11-1 (AAC38151), Bacillus halodurans (BAB05967), Deinococcus radiodurans (AAF10396), M.tuberculosis (LipH) (Z95586), and M.tuberculosis (LipQ) (D70868).

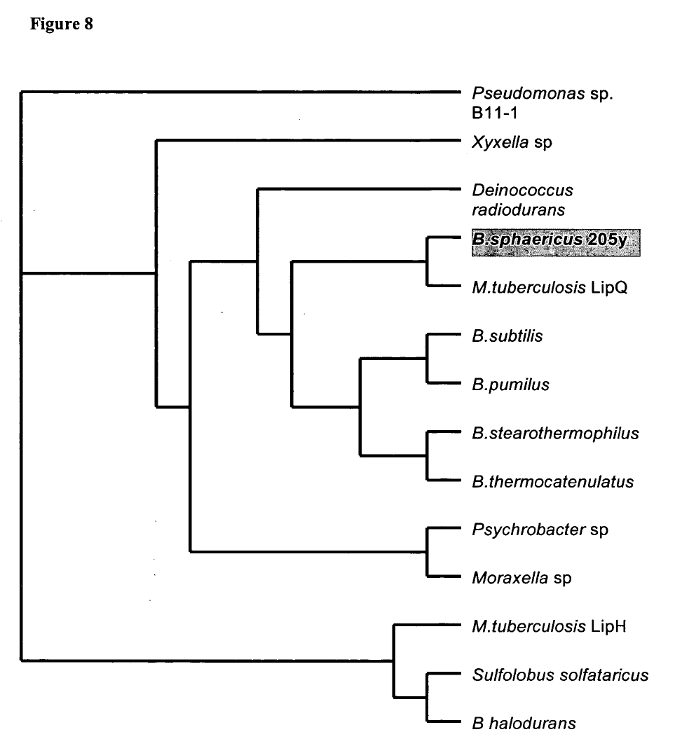

FIG. 9: SDS-PAGE of total cellular proteins stained for protein in (12% w/v). E.coli cells harbouring the pUC 19 (control) and placLIP were grown in LB broth containing 200 μg of ampicillin to A₆₀₀ 0.5-0.6. Then IPTG (1 mM) was added to the culture to induce expression. A 1 ml sample of the culture broth was centrifuged for 10 min and bacterial cells resuspended in 100 μl Laemmli sample buffer. Aliquots of 15 μl were subjected to SDS-PAGE. The gels were stained with Coomassie Brilliant blue. Lanes 1 and 8: protein marker (kDa), Lane 2-4: Samples [E.coli (pUC 19)] of 1 h, 2 h and 3 hour respectively after addition of IPTG, Lane:5-7: Samples [E.coli (placLIP)] of 1 hour, 2 hour and 3 hour respectively after addition of IPTG.

### BRIEF DESCRIPTION OF THE SEQUENCES

SEQ ID NO:1 is a degenerate forward primer used to amplify the 16S rDNA sequence.

SEQ ID NO:2 is a degenerate reverse primer used to amplify the 16S rDNA sequence.

SEQ ID NO:3 is the nucleotide sequence assigned the accession number AF435435.

SEQ ID NO:4 is the lipase gene sequence of Bacillus sphaericus 205y organic solvent-tolerant with assigned accession number AF 453713.

SEQ ID NO:5 is the amino acid sequence encoded by the lipase gene sequence of Bacillus sphaericus 205y organic solvent-tolerant with assigned accession number AF 453713.

SEQ ID NO:6 is the 22 PELE-Forward primer used in amplification of the gene.

SEQ ID NO:7 is the 22 His-Reverse primer used in amplification of the gene.

SEQ ID NO:8 is the conserved pentapeptide, Gly-X-Ser-X-Gly, contained in the presumed catalytic region of the lipase from Bacillus sphaericus 205y.

SEQ ID NOs:9-15 are seven major hydrophobic grooves of the subject nucleotide sequence: residues 13-35 (SEQ ID NO:9), 44-73 (SEQ ID NO:10), 74-99 (SEQ ID NO:11), 160-169 (SEQ ID NO:12), 214-222 (SEQ ID NO:13), 244-253 (SEQ ID NO:14) and 268-276 (SEQ ID NO:15).

SEQ ID NO:16 is the conserved pentapeptide, Ala-X-Ser-X-Gly, that was found in the Bacillus species.

Isolation of Organic Solvent-Tolerant Strain.

Isolation and Identification of Bacillus sphaericus 205y strain form the basis of the present invention of organic solvent-tolerant strain. The Bacillus sphaericus 205y culture could be obtained from Enzyme and Microbial Technology Research Department of Biochemistry and Microbiology, Faculty of Science and Environmental Studies, University Putra Malaysia. One gram of soil is resuspended in 10 ml basal mineral agar supplemented with selected hydrocarbon (0.1% v/v). The basal media contained (w/v) K₂HPO₄ 0.5%, NH₄Cl (1.0%), Na₂SO₄ (2%), KNO₃ (2%), MgSO₄.7H₂O (0.2%).

Selected hydrocarbons would either be benzene, toluene or mixture of both as their sole carbon and energy source. Pure isolates were prescreened in media spiked with 1% v/v hydrocarbon. Isolates were grouped into groups of good, moderate, acceptable and negative, based on the turbidity of the media (Inoue et al., 1989). Non-inoculated controls were also used to ensure that the turbidity is due to growth.

Isolate 205y is able to grow at high concentration of solvents up to 75% (v/v). Besides benzene and toluene, 205y also exhibited good growth in ethylbenzene and ρ-xylene, (Table 1).

TABLE 1
Growth of isolate 205y in different organic solvents and
concentrations.

| | Hydrocarbon (v/v) | | | | | |
|---|---|---|---|---|---|---|
| | 1% | 5% | 10% | 25% | 50% | 75% |
| | Benzene | | | | | |
| Growth of isolate 205y | ++ | +++ | +++ | ++ | ++ | ++ |
| | Toluene | | | | | |
| | +++ | +++ | ++ | ++ | ++ | ++ |
| | Ethylbenzene | | | | | |
| | ++ | ++ | +++ | ++ | ++ | ++ |
| | ρ-Xylene | | | | | |
| | ++ | +++ | +++ | +++ | ++ | ++ |

Growth was visually determined
(+) Acceptable;
(++) Moderate;
(+++) Good

Screening and Lipase Production

Bacillus sphaericus 205y is able to grow at high concentration of solvent screened for lipase activity on triolein plates (Samad et al., 1989). In order to select the best lipase production in liquid medium strains were cultivated in M1 (w/v): peptone 3%, yeast extract 1%, NaCl 0.5%, olive oil 1% (v/v). Lipase production of Bacillus sp 205y was also tested in GYP (w/v): Glucose 2%, yeast extract 1%, peptone 1%, CH₃COONa.3H₂O, 1%, MgSO₄.7H₂O 0.03%, MnSO₄ 0.01%, KCl 0.05%, olive oil 2%(v/v), pH 7.0, M3 (w/v): Nutrient broth 0.325%, gum arabic 1%, CaCl₂.2H₂O 0.05%, Tween 80 1% (v/v), olive oil 1% (v/v); M5: Nutrient broth 0.8% (w/v) (Oxoid), triolein 1% (v/v), pH 7.0;

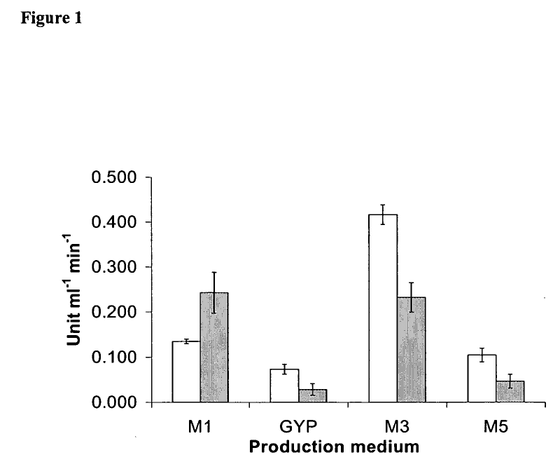

Lipase production by isolate 205y in M3 is significantly higher than other production media. Maximum lipase production (0.40 Uml⁻¹ min⁻¹) is observed in M3 after 24 hours incubation (FIG. 1). This may be due to the presence of Tween 80 and gum arabic in M3. Tween 80 may have induced the production of lipase (Fakhreddine et al., 1998 had reported that removal of Tween 80 from production medium resulted in loss of all the ρ-nitrophenyl caprylate (pNPN16) which demonstrated the induction effect by this substrate for lipase activity). It is also reported that gum arabic releases cell surface-bound lipase, therefore, resulting in higher activity.

Lipase Assay

Lipase activity is measured with olive oil emulsion as substrate (Kwon and Rhee. 1991). All reactions were performed at 37° C. for 15 minutes at shaking condition of 200 rpm unless otherwise indicated. One unit of lipase activity was defined as 1.0 μmol of fatty acid liberated per min per ml.

Identification and Taxonomical Studies

The isolated strain was deposited at the DSHZ and was assigned Acession No. DSH17161. The isolated strain was identified according to method described in “Bergey's Manual of Determinative Bacteriology” and also via 16S rDNA sequence. 16S rDNA sequence was amplified via PCR with two degenerate primers:

Forward:

(SEQ ID NO:1)

5′-CCGAATTCGTCGACAACAGAGTTTGATCCTGGCTCAG-3′;
and
Reverse:

(SEQ ID NO:2)

5′-CCCGGGATCCAAGCTTACGGCTACCTTGTTACGACTT-3′.

These primers amplified the 1500-bp PCR product. Genomic DNA of Bacillus sphaericus 205y is added to PCR reactions containing Forward and Reverse primers. After 3 minutes at 94° C., 30 PCR cycles (94° C. for 1 minute, 58° C. for 2 minute and 72° C. for 2 minute) were performed. This is followed by 1 cycle of 7 minutes at 72° C. and hold at 4° C. The amplified products were examined by electrophoresis and reaction products were ligated into T/A cloning vector (Invitrogen) according to manufacturer's instructions.

After transformation into E.coli, plasmids were extracted and sequenced. A homology search was performed with Genbank database. For further characterization of strain 205y, we constructed a phylogenetic tree based on comparison of 16S rDNA sequence of this strain and those of type strains of Bacillus species.

The microbiological characteristics of strain 205y showed circular, smooth, convex, entire and opaque on nutrient agar. The bacterium is a gram-positive rod, formed spore and occurred singly and in pairs. It is able to hydrolyse gelatin, casein, triolein and produces gas and acid in glucose and sucrose broth. Strain 205y is also able to reduce nitrate to ammonia. A typical characteristic of the Bacillus genus is shown in Table 2.

TABLE 2
Biological characteristics of the isolate 205y.

| Characteristic | 205y result |
|---|---|
| Morphological | |
| Shape | Rods |
| Gram stain | Positive |
| Spore | + |
| Spore position | Terminal |
| Motility | + |
| Culture | |
| Growth at: | |
| Room temperature | + |
| 37° C. | + |
| 45° C. | + |
| Biochemical | |
| Denitrification | + |
| Oxidase test | − |
| Catalase test | + |
| Hydrogen sulfide | − |
| formation | |
| Methyl red test | + |
| Vogue-Proskauer test | − |
| Argininedihydrolase test | − |
| Indole production | − |
| Urease activity | V |
| Citrate utilization | + |
| Hydrolysis of: | |
| Tributyrin | + |
| Triolein | + |
| Gelatin | + |
| Tween 80 | + |
| Starch | − |
| Casein | + |
| Carbon sources for growth | |
| L-Arabinose | − |
| D-Glucose | + |
| D-Xylose | + |
| Raffinose | − |
| Lactose | − |
| Sucrose | + |
| Rhamnose | − |
| D-Sorbitol | − |
| m-Inositol | − |
| D-Mannitol | + |
| Adonitol | − |
| Malonate | − |

Note:
+ positive results,
− negative results,
V variable results

The nucleotide sequence described in this paper has been deposited into Genbank data library and assigned the accession number AF435435.

(SEQ ID NO:3)

gccctcgagtttgatcctggctcaggacgaacgctggcggcgtccctaat
acatgcaagtcgagcgaacagaaaaggagcttgctcctttgacgttagcg
gcggacgggtgagtaacacgtgggcaacctaccttatagtttgggataac
tccgggaaaccggggctaataccgaataatctatttcacttcatggtgaa
atactgaaagacggtctcggctgtcgctataagatgggcccgcggcgcat
tagctagttggtgaggtaacggctcaccaaggcgacgatgcgtagccgac
ctgagagggtgatcggccacactgggactgagacacggcccagactccta
cgggaggcagcagtagggaatcttccacaatgggcgaaagcctgatggag
caacgccgcgtgagtgaagaaggttttcggatcgtaaaactctgttgtaa
gggaagaacaagtacagtagtaactggctgtaccttgacggtaccttatt
agaaagccacggctaactacgtgccagcagccgcggtaatacgtaggtgg
caagcgttgtccggaattattgggcgtaaagcgcgcgcaggcggtccttt
aagtctgatgtgaaagcccacggctcaaccgtggagggtcattggaaact
gggggacttgagtgcagaagaggaaagtggaattccaagtgtagcggtga
aatgcgtagagatttggaggaacaccagtggcgaaggcgactttctggtc
tgtaactgacgctgaggcgcgaaagcgtggggagcaaacaggattagata
ccctggtagtccacgccgtaaacgatgagtgctaagtgttagggggtttc
cgccccttagtgctgcagctaacgcattaagcactccgcctggggagtac
ggtcgcaagactgaaactcaaaggaattgacgggggcccgcacaagcggt
ggagcatgtggtttaattcgaagcaacgcgaagaaccttaccaggtcttg
acatcccgttgaccactgtagagatatggttttcccttcggggacaacgg
tgacaggtggtgcatggttgtcgtcagctcgtgtcgtgagatgttgggtt
aagtcccgcaacgagcgcaacccttgatcttagttgccatcatttagttg
ggcactctaaggtgactgccggtgacaaaccggaggaaggtggggatgac
gtcaaatcatcatgccccttatgacctgggctacacacgtgctacaatgg
acgatacaaacggttgccaactcgcgagagggagctaatccgataaagtc
gttctcagttcggattgtaggctgcaactcgcctacatgaagccggaatc
gctagtaatcgcggatcagcatgccgcggtgaatacgttcccgggccttg
tacacaccgcccgtcacaccacgagagtttgtaacacccgaagtcggtgg
ggtaacctt.

Effect of Organic Solvents on Lipase Activity

Bacillus sphaericus 205y is grown at 37° C. in M3 medium. The culture medium (24 h) is centrifuged at 12,000 rpm at 4° C. for 15 minutes by using membrane filter (0.25 μm pore size) as crude enzyme. Investigation of various effects of organic solvents at the concentration of 25% (v/v) (1 ml of organic solvent/3 ml of crude enzyme) on the lipase activity was done. The reaction mixture was incubated for 30 minutes at 37° C. under shaking condition (200 rpm). New organic solvent-tolerant B. sphaericus strain 205y that is capable of growing in the presence of BTEX (Benzene, Toluene, Ethylbenzene, ρ-Xylene) was isolated. A new strain of Bacillus sphaericus 205y that is stable in solvent and in addition produce an organic solvent-stable lipase was isolated.

Cloning and Expression of Novel Lipase Gene from Bacillus sphaericus 205y

The E. coli strains used in DNA manipulations were: Top 10 (F⁻ mcrA Δ (mrr-hsdRMS-mcrBC) φ80lacZΔM15 ΔlacX74 recA1 deoR araD139 (ara-leu) 7697 galU galK rpsL (Str^{R}) endA1 nupG), and BL21 (F⁻ ompT hsdSB (r_{B}⁻m_{B}) gal dcm). E. coli BL21 is used to overexpress the lipase gene. All E. coli strains were cultivated in Luria-Bertani medium (tryptone (1% w/v), yeast extract (0.5% w/v), NaCl (1% w/v) per liter of deionized water, ampicillin (100 μg/ml) pH7.0 at 37° C.

Recombinant DNA Techniques

Methods for DNA manipulation were used as described by Sambrook et al., 1989. Restriction enzymes and other modifying enzymes was used according to manufacturer recommendation (Promega, USA). Chomosomal DNA from Bacillus sphaericus is extracted and partially digested with Sau 3Al. The DNA fragment in the range of 2 kilobase and 10 kilobase was excised and purified using Qiaex II extraction kit (Qiagen, Germany) according to the manufacturer's instructions. Genomic library was constructed by using pUC 19, which was previously digested with Bam HI.

Screening of Positive Recombinant Clone

Recombinant clones were screened for lipase activity on tributyrin and triolein agar plates (Samad et. al.,1989). Lipase will produce a halo on tributyrin agar and blue zone on triolein agar. Recombinant clones were also screened with Rhodamine B agar (Kouker and Jaeger, 1987).

Lipase Assay

All positive recombinant clone isolated from the triolein plate were cultivated in Lubria-Bertani medium and assayed for lipase activity after 24 hour and 48 hour incubation. Lipase activity was measured with colorimetric method developed by Kwon and Rhee, (1986). The standard curve of free fatty acid must first be determined with pure oleic acid. Samples containing 2.0-50.0 μmole oleic acid were dissolved in test tubes containing 5 ml of isooctane. Cupric acetate-pyridine reagent (1 ml) is added and the two phases were mixed vigorously for 90 seconds using a vortex mixer.

The mixture is allowed to stand still for 20-30 seconds until the aqueous phase was sedimented clearly from the solution of isooctane and fatty acids. The reaction mixture contains 0.01 M calcium chloride (20 μl), olive oil emulsion (2.5 ml) and crude enzyme (1 ml). The reaction mixture was continuously agitated by using a horizontal shaker at 200 rpm at 37° C. The reaction was stopped by adding isooctane (5 ml) to dissolve free fatty acids produced by olive oil hydrolysis. Lipase activity was determined by measuring the free fatty acids from the standard curve of free fatty acids. One unit of lipase activity was defined as 1.0 μmol of fatty acid liberated per min per ml.

Sequencing and Analysis of Lipase Gene

Samples were sequenced using an ABI PRISM 377 Genetic Analyzer (Perkin-Elmer). Analysis of the sequence and database similarity search is done using National Centre of Biotechnology and Blocks database at Fred Hutchison Cancer Research Center (USA). Analysis of the lipase gene was done with Biology Workbench and Expasy Molecular Biology Server. The lipase gene sequence of Bacillus sphaericus 205y organic solvent-tolerant has been deposited to EMBL/DDBJ/GenBank data library with assigned accession number AF 453713 (SEQ ID 4 and SEQ ID 5).

Nucleotide Sequence SEQ ID 4
gtgaccaccccgtagctatgattaatggttgcgggtattttttgtaatat
taagtgtgatttgtgcgagtagaagtaacatatggtgacaaaattgaaac
tactagatgaactttgggctgcatttttaaatgggaatgataataacata
tcgtatgtgttaaaccgtcgaacgggtgaagttttttgaaataaaggagg
tattaaaaagatgaatcagataacaaattggcgacctagaggttttcttc
agtggctactcacaatcctctctgtgatcgtatgttttataatcattata
gtggcatactatatttttaatccaagtaatatggataaaataatgagtct
ccttgcatgggggacttctatatttccgattatgctagtagttacagcat
tcataataatcgttttactagctctttctttttggaagaaaacattcatt
gctcttacagtgttatttcctattttgttgttattaatgtttttaactgt
gcagccaattagtactatgaaaagctatgctaaaagcgagaatgtatcgg
ttgctcttagctcacattttttctataaccaaaatatctcaacaaaacct
agtgtagatgtggtgtatgggaaaactacggacggcattgaactaaagct
tgatgtgtggccagcaaagaaaaaatcagaggatgttttaacacctgtct
tgttcaagtgcatgggggtggttgggtaagtggagataaaggccaagtgc
aagactggaatcagtggatgaatgatcaagggtacactgtttttgatgtc
caataccgtatgcctcctgtagcaggctggaaagatgaagtgggggatgt
taaatcagcgattggttggattgtgcaacatgcagatacgtataagattg
atccgaacagaatcattttaatgggagagtctgctgggggaaatctagct
atgcttgctgcctatagtttgggagataagcatttaccgccatcaacgga
tgttccggatgtaccgatcaaggcagtgattaacatgtatgggccgtcag
atatgactgcgttttacaagaacaatccaagtaagcgttatgtccaagat
gttctggatcagtacattggaggttcaccatccgattaccctgcgcgtta
taagaagctttcccctataagctatatccaggaacatacgccaccaacta
ttatgtttttagggacgggtgatcgtattgttcctgtagagcaggcaaac
gtattggatgacaaactgacgacaagcggtgtagcgcatgaactttatct
tcttccaaaggtcgatcatggctttgatgcaaatccaggtagtttgagca
cgcaatttgccaaggagaaagtaaaagcatttcttcaaaaatataataaa
taatcattatgcaatagaaaaacctttgaagacattaaccttcaaaggct
ttttttagcaagtacatgtagaatttattatccgtcccaaagtatatatg
aaaatgcttactgcaatttggattggaaagagtatgacaatgcttacagt
ttttactat
Amino acid SEQ ID 5
MNQITNWRPRGFLQWLLTILSVIVCFIIIIVAYYIFNPSNMDKIMSLLAW
GTSIFPIMLVVTAFIIIVLLALSFWKKTFIALTVLFPILLLLMFLTVQPI
STMKSYAKSENVSVALSSHFFYNQNISTKPSVDVVYGKTTDGIELKLDVW
PAKKKSEDVLTPVIVQVHGGGWVSGDKGQVQDWNQWMNDQGYTVFDVQYR
MPPVAGWKDEVGDVKSAIGWIVQHADTYKIDPNRIILMGESAGGNLAMLA
AYSLGDKHLPPSTDVPDVPIKAVINMYGPSDMTAFYKNNPSKRYVQDVLD
QYIGGSPSDYPARYKKLSPISYIQEHTPPTIMFLGTGDRIVPVEQANVLD
DKLTTSGVAHELYLLPKVDHGFDANPGSLSTQFAKEKVKAFLQKYNK

Expression of Lipase Gene

The primers used in amplification of the gene were 22 PELE-Forward 15′-GGCGGAGGTATGAATTCGATGAATCAGATAACAAAT-3′ (SEQ ID NO:6) and 22 His-Reverse 5′-AGGTTAAGTCTTCAA-GTTGTCGACTGCATMTGATT-3′ (SEQ ID NO:7). The underlined sequences indicate the restriction sites for Eco RI (GAATTC) and Sal I (GTCGAC). The 100 ρl reaction mixture is subjected to 3 minutes at 94° C. (initial denaturation), followed by 30 cycles of 94° C. at 1 min, 58° C. 2 min and 72° C. 2 min; and 7 minutes at 72° C. The PCR reaction is done in a thermocycler (GeneAmp PCR system 2400, Perkin Elmer, Foster, Calif.).

Detection of Lipase via SDS-PAGE

Cells containing the lipase gene were propagated in 100 ml of LB-broth supplemented with (200 μg/ml) of ampicillin at 37° C. with shaking at 200 rpm. When the culture reached A₆₀₀ of 0.5-0.6, IPTG (1 mM) was added to induce expression. The cells were incubated further for 4 hour. Cells (1 ml) are taken out for SDS-PAGE analysis with discontinuous buffer system at 0, 1, 2, 3 and 4 hour. After electrophoresis, the gel is stained with Coomassie Brilliant Blue R-250 [0.5% (w/v); in 25% (v/v) isopropanol and 10% (v/v) acetic acid] for 30 minute at room temperature with gentle agitation and destained with solution containing methanol [10% (v/v)] and acetic acid [10% (v/v)] for 1 hour.

Recombinant Enzyme Assay and Organic Solvent-Tolerant Test

After induction with IPTG (1 mM), 10 ml of cells were pelleted by centrifugation at 10,000 gram and resuspended in 10 ml of Phosphate buffer (PBS). The cells were mixed thoroughly, placed on ice, and sonicated with Branson sonicater (2 min, 30% duty cycle). The broken cells were pelleted and the supernantant were used in determining the lipase activity and organic solvent tolerant. Various organic solvents were tested at the concentration of 25% (v/v) by addition of 1 ml of organic solvent to 3 ml of crude enzyme. The reaction mixture was incubated for 30 minutes at 37° C. under shaking condition (200 rpm). Remaining activity was assayed under standard condition and expressed as the percent of the control value. For control, distilled water was added instead of solvent. The organic solvents used were hexane, ρ-xylene, benzene, dimethy sulfoxide, acetonitrile and hexadecane. Each experiment was done in triplicate.

Lipase Gene Isolation via Genomic Library

Bacillus sphaericus is so far known to be associated with production of binary toxin, which is used in mosquito larvae control. The Bacillus sphaericus 205y lipase gene was isolated via genomic DNA library strategy with direct selection method. A genomic library is a collection of clones sufficient in number to be likely to contain every single gene present in a particular organism (Brown, 1990). Extracted genomic DNAs were partially digested with Sau 3Al to generate a range of fragmented DNAs desired for genomic library cloning. Optimisation was done on a small-scale partial digestion in order to estimate the concentration of Sau 3Al needed to generate the desired DNA fragments.

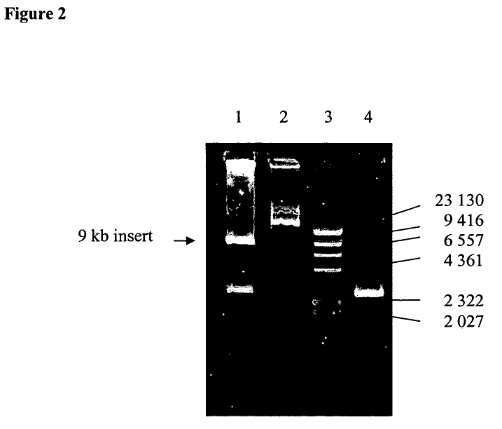

Through genomic library, about 9000 recombinant clones was produced. Four clones were positive on tributyrin-amp agar. Out of these, one produced blue zone on triolein-amp Victoria Blue agar and orange-fluorescent on triolein-amp Rhodamine B agar. This clone was found to contain a 9 kilobase DNA fragment after gel electrophoresis (FIG. 2). The 9 kilobase fragment was purified and partially digested with Sau 3Al again to generate another genomic library. From the second genomic library 4000 recombinant clones were produced with one recombinant clone containing a 2.9 kilobase insert produced halo on tributyrin-amp agar and also produced blue zone on triolein-amp victoria blue agar. Grown on triolein-amp Rhodamine B agar, this recombinant clone produced an orange fluorescent under ultraviolet radiation. This clone was regarded as 205y PLIP. The recombinant clone is grown in LB-amp and lipase assay is measured after 24 hour incubation at 37° C. with shaking at 200 rpm. However the activity produced by this clone was low (0.01 U ml⁻¹ min⁻¹).

Restriction Mapping of Putative Lipase Gene

The recombinant plasmid was digested with several restriction enzymes (RE) to determine the restriction site in the insert. Eco RI, Xba I, Hind III, Sac I, Kpn I, Sma I Bam HI, and Bgl I. Digestion with Eco RI, Kpn I, Sma I, Xba I and Bam HI produced a linear band on agarose gel (1% w/v) after gel electrophoresis. Therefore these RE sites were not present in the insert.

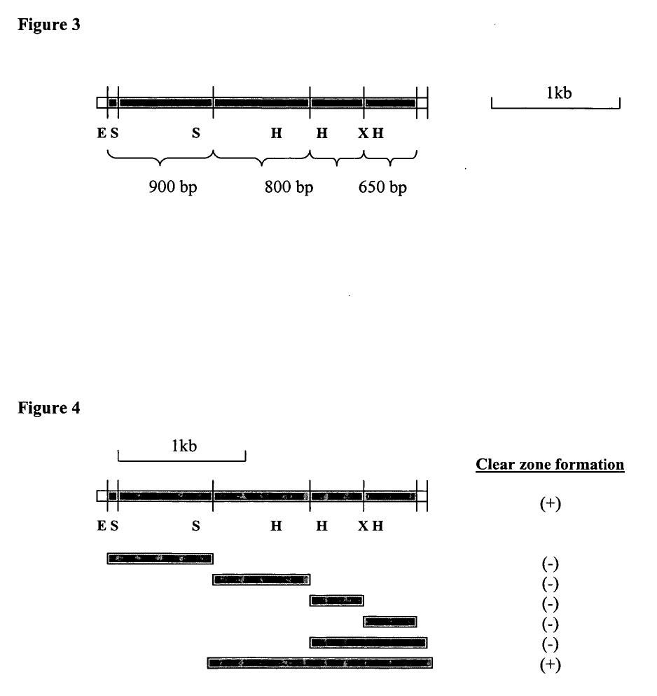

Digestion with Sac I produced two fragments (900 basepair and 4400 basepair) and Hind III produced three bands (650 basepair, 550 basepair and 4200 basepair). Combination of these enzymes produced four bands (900 bp, 800 bp, 650 basepair, 550 basepair and 2700 basepair). Based on this information a restriction map was plotted to assist further subcloning experiments (FIG. 3). Each of the fragments were isolated and cloned into pUC 19 individually.

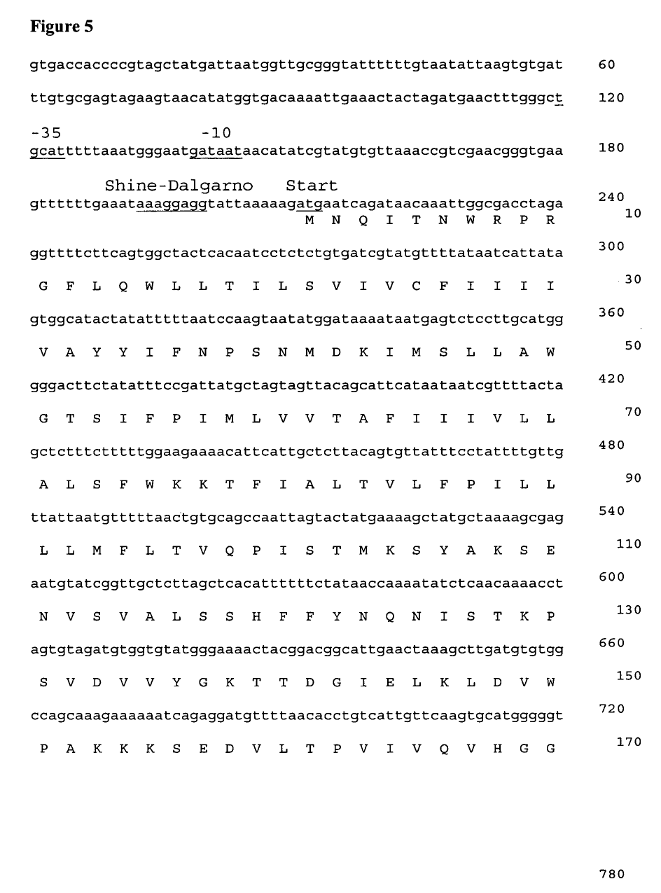

Recombinant clones carried the 900 bp, 800 bp, 650 bp, and 550 bp did not produce halo on tributyrin-amp plate and thus did not contain or partially contain the lipase gene. However recombinant clone transformed with plasmid that carried the fragments in between the Sac I and Hind III site (˜2 kilobase) (FIG. 4) produced positive result not only in tributyrin-amp plate but also produced blue zone on triolein-amp Victoria Blue agar plate. This plasmid was regarded as pLIP2 and was isolated and sequenced.

The nucleotide sequence of the region of pLIP2 (˜1.7 kb) was determined and was found to contain a single open reading frame. The Sac I and Hind III region were initially sequenced with M13 forward and M13 reverse primers. Subsequently primers were designed to complete the sequencing.

Nucleotide Sequence Analysis

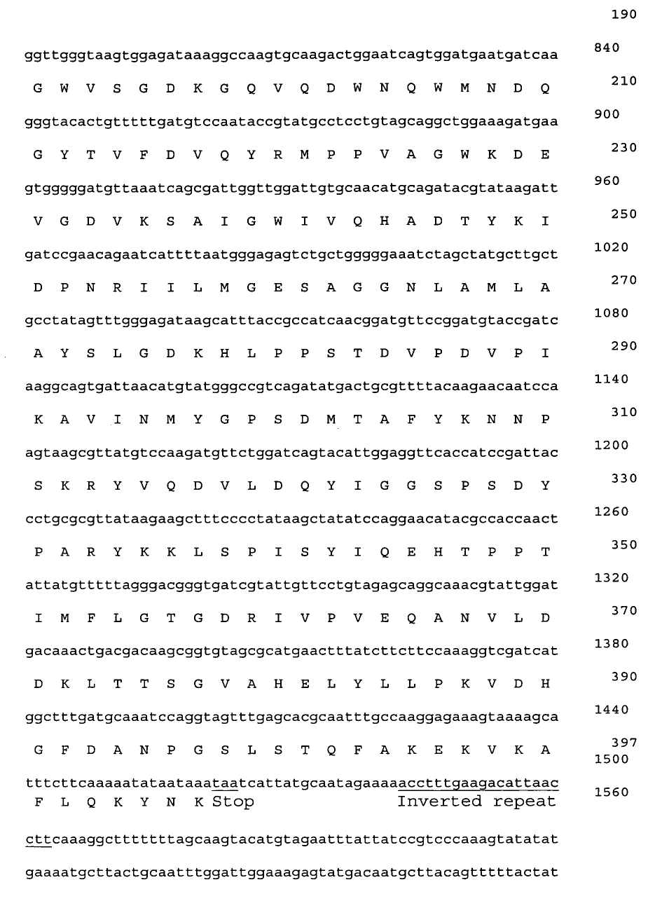

The entire 1.7 kilobase containing the putative lipase gene was sequenced and was found to contain a single open reading frame (ORF) comprising of 1191 basepair extending from 210 to 1401 (FIG. 5). Putative −35 (CTGCAT) and −10 (GATAAT) promoter sequences were observed as well as a stem loop structure downstream from the stop codon TAA.

The ORF have six possible methionines as start codon at position 211, 331, 343, 382, 487 and 514. The ATG initiation codon at position 211 is the most likely to be the start codon since the AAAGGAGG sequence was found 10 bp upstream and corresponded to the concensus ribosome binding site of Bacillus sphaericus 205y. The primary structure of the lipase deduced from the nucleotide sequence showed that lipase is composed of 397 amino acid residues. The preprotein molecular mass calculated from the expected amino acids sequence is 44441.6 Da.

In addition, a possible signal peptide sequence was detected in the first 32 amino acid residues (Met¹-Ala³²). The putative signal peptide was in agreement with the rules deduced from known proteins with signal peptides. Gierach (1989) has stated that the signal peptide is usually composed of 13 to 36 amino acids having at least one positively charged residue followed by a hydrophobic core of 10 to 15 residues and ended with a small, neutral residue, often Ala. The presumed putative −35 and −10 sequences, CTGCAT and GATAAT, respectively, resemble the consensus sequences for the promoter region recognised by σ⁴³ RNA polymerase of Bacillus subtilis, which has been reported to be TTGACA and TATAAT (Dartois et al. 1992).

The Bacillus sphaericus 205y lipase gene (BS-LIP) shared little homologies with other lipases. However the overall amino acid homology near the region of the active pentapeptide site showed significant homology (Table 3). The results suggested that the lipase produced by Bacillus sphaericus 205y was not closely related to any of the lipases previously characterised. It is well known from numerous sequence comparisons that lipases share very little homologies except within the presumed catalytic region containing the conserved pentapeptide, Gly-X-Ser-X-Gly (SEQ ID NO:8). Interestingly, the highest similarity of the BS-LIP gene was found towards an ORF in the genome of the bacterial pathogen Mycobacterium tuberculosis-Lip Q (18%).

Conserved lipase motifs showed that this unknown protein was likely to be a lipase. However, the functions of this hypothetical lipase have not yet been clarified. Therefore it remains to be tested whether the high similarities between the lipases of these two organisms reflect a similar enzyme activity.

TABLE 3
Comparison of amino acid composition of lipases from various
organisms. The sequences were obtained from GenBank. The abbreviation
used were 205y: Bacillus sphaericus 205y (AF 453713), X. sp: Xyxella sp.
(XF1253), S. so: Sulfolobus solfataricus (AAK42652), P. sp.: Psychrobacter sp
(AAF70342), M. sp: Moraxella sp. (P24484), B11-1: Pseudomonas sp B11-1
(AAC38151), B. ha: Bacillus halodurans (BAB05967), D. ra: Deinococcus
radiodurans (AAF10396), LipH: M. tuberculosis (LipH) (Z95586), and LipQ:
M. tuberculosis (LipQ) (D70868).

| Residues | 205y | X. sp | S. so | P. sp | M. sp | B11-1 | B. ha | D. ra | LipH | LipQ |
|---|---|---|---|---|---|---|---|---|---|---|
| Ala | 23 | 39 | 14 | 39 | 50 | 46 | 39 | 53 | 44 | 67 |
| Arg | 7 | 14 | 13 | 11 | 13 | 20 | 16 | 44 | 17 | 35 |
| Asn | 17 | 15 | 22 | 12 | 21 | 8 | 11 | 25 | 7 | 10 |
| Asp | 25 | 18 | 19 | 28 | 26 | 22 | 25 | 17 | 26 | 21 |
| Cys | 1 | 1 | 1 | 10 | 8 | 7 | 0 | 10 | 1 | 5 |
| Gln | 17 | 16 | 7 | 17 | 25 | 18 | 14 | 14 | 4 | 11 |
| Glu | 9 | 16 | 14 | 16 | 19 | 18 | 18 | 11 | 15 | 15 |
| Gly | 25 | 29 | 19 | 30 | 26 | 20 | 28 | 36 | 27 | 35 |
| His | 7 | 8 | 8 | 21 | 17 | 6 | 12 | 8 | 8 | 14 |
| Ile | 32 | 15 | 22 | 22 | 19 | 7 | 25 | 13 | 11 | 19 |
| Leu | 36 | 29 | 29 | 46 | 52 | 39 | 30 | 43 | 36 | 33 |
| Lys | 28 | 15 | 19 | 19 | 21 | 2 | 12 | 12 | 3 | 6 |
| Met | 13 | 6 | 9 | 7 | 6 | 7 | 10 | 7 | 6 | 11 |
| Phe | 17 | 11 | 11 | 11 | 9 | 15 | 18 | 14 | 7 | 14 |
| Pro | 25 | 16 | 17 | 21 | 25 | 19 | 26 | 41 | 29 | 32 |
| Ser | 28 | 14 | 20 | 24 | 26 | 14 | 27 | 32 | 12 | 33 |
| Thr | 22 | 21 | 14 | 23 | 25 | 10 | 16 | 20 | 19 | 14 |
| Trp | 10 | 5 | 2 | 5 | 6 | 3 | 4 | 10 | 6 | 9 |
| Tyr | 18 | 8 | 24 | 14 | 14 | 10 | 19 | 12 | 10 | 12 |
| Val | 37 | 29 | 27 | 24 | 25 | 17 | 35 | 32 | 31 | 25 |
| Total | 397 | 325 | 311 | 400 | 433 | 308 | 385 | 454 | 319 | 421 |

Amino Acid Composition

Out of the 397 amino acid residues deduced from the nucleotide sequence of Bacillus sphaericus 205y, 34 residues were negatively charged (Asp and Glu) and 35 were positively charged (Arg and Lys). The total charged residues (Arg, Glu, Lys, Arg, His) is 76 amino acids or 19.1% of the total amino acids. Total hydrophobic residue (Ala, Ile, Leu, Met, Phe, Pro, Trp, Val) is 193 amino acids (48.6%) and total uncharged residue (Asn, Cys, Gln, Gly, Ser, Thr, Tyr) is 128 amino acids (32.2%). The amount of polarity plays an important role in the solubility of the enzyme in which the polar amino acids may interact with water molecules. The lipase gene (BS-LIP) has instability index of 29.45, which was considered stable according to Guruprasad et al., 1990. An enzyme was considered unstable when the instability index is more than 40. The deduced amino acid of BS-LIP showed an aliphatic index of 99.62. According to Ikai (1980) aliphatic index is defined as relative volume of a protein occupied by aliphatic side chains (Ala, Val, Ile, and Leu) of proteins.

The aliphatic index is regarded as a positive factor for the increased stability of the protein. From the amino acids comparison study of ten lipases, it showed that Ala, Leu and Val were abundant in these lipases (Table 4). Val is most abundant in BS-LIP. On the other hand, residues like Cys, His, Met, Trp and Phe were less abundant. All lipases have shown a low percentage of Cys molecules if not any. Low numbers of Cys residue are common in lipases. Proteins lacking cysteine or with a low content of cysteine are generally more flexible molecules whose tertiary structure relies on weaker bonds. It has been noted that many extracellular bacterial proteins contain a low level of cysteine (Dartois et al. 1992).

Hydrophobicity Profile and Signal Peptide Prediction

The hydrophobicity of the lipase gene from Bacillus sphaericus 205y was determined with ProtScale tools of the Expasy Molecular Biology with Kyte and Doolittle method. Based on amino acid sequence, it was shown that the region of hydrophobic and hydrophilic ratio was approximately the same.

Hydrophobicity of a protein is a significant factor in identifying signal sequences and also affect the stability of the protein folding (Henrissat, 1992). There are seven major hydrophobic grooves, which were residues 13-35 (Q-W-L-L-T-I-L-S-V-I-V-C-F-I-I-I-I-V-A-Y-Y-I) (SEQ ID NO:9), 44-73 (I-M-S-L-L-A-W-G-T-S-I-F-P-I-M-L-V-VT-A-F-I-I-I-V-L-L-A-L-S) (SEQ ID NO:10), 74-99 (F-W-K-K-T-F-I-A-L-T-V-L-F-P-I-L-L-L-L-M-F-L-T-V-Q-P-I) (SEQ ID NO: 11), 160-169 (T-P-V-I-V-Q-V-H-G) (SEQ ID NO:12), 214-222 (V-K-S-A-I-G-W-I-V) (SEQ ID NO:13), 244-253 (G-N-L-A-M-L-A-A-Y-S) (SEQ ID NO:14) and 268-276 (V-P-I-K-A-V-I-N-M) (SEQ ID NO:15). The first region was predicted to be signal peptide. The hydrophobicity regions of 214-222 and 244-253 that were flanking the catalytic Ser-240 probably act as a lid and to govern the interaction of enzyme at polar/non-polar interface.

DNA Sequence Homology with B. subtilis and B. pumilis Lipase Gene

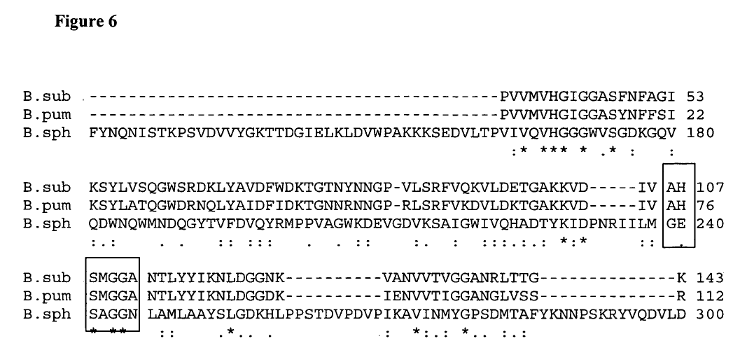

Homology search was performed with BLAST and PSI-BLAST. Results showed that the region have similarity of 27% (80/397 amino acid) with B. subtilis and B. pumilis (FIG. 6). Bacillus subtilis and Bacillus pumilis lacks of the Gly-X-Ser-X-Gly conserved pentapeptide among lipases and have Ala replacing the first Gly. Lipase from Bacillus sphaericus 205y however has the common conserved pentapeptide which have been purported to play essential role in catalysis. Based on the 16S rDNA sequence phylogenetic study, Bacillus sphaericus 205y is distantly related to other Bacilli species. Therefore the lipase secreted by this bacteria may have a different evolutionary path, which explained why the alanine replacement of the first glycine did not take place.

DNA Sequence Homology with Others Microbial Lipases Gene

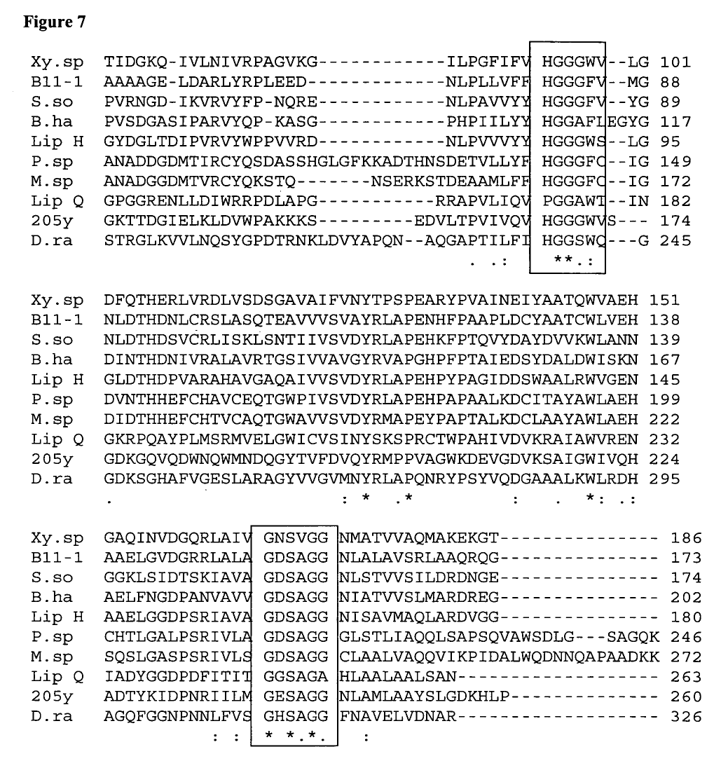

Deduced amino acid of lipase from Bacillus sphaericus 205y lipase was compared with ten other lipases that obtained from the NCBI database showed high similarities to several lipases in the conserved regions (Gly-X-Ser-X-Gly). However, the overall similarities were low when compared to most lipases including those from Bacillus sp (10%). In contrast to low overall homology, the Bacillus sphaericus 205y lipase has highest homology to a putative lipase of Mycobacterium tuberculosis LipQ (18%) especially near the conserved active site motif. This protein contained typical lipase motifs and was therefore likely to be a lipase. The Bacillus sphaericus 205y lipase also has the His-Gly dipeptide, which was found in most Gly-X-Ser-X-Gly (SEQ ID NO:8) lipases (FIG. 7). A tree was constructed by neighbour-joining method to reveal relationship between the different members of lipases. The lipases of mesophilic Bacillus i.e. Bacillus subtilis and Bacillus pumilis and thermophilic Bacillus i.e. Bacillus thermocatenulatus and Bacillus sterothermophilus have higher homology to each other than with B. sphaericus 205y lipase.

All lipases can be divided into two distinct classes on the basis of the codon for active site serine, which can be either AGY or TCN (Sullivan et. al. 1999). It has been suggested that codon usage for active site serine can be used for studying the evolutionary origin of different lipases. The lipase from B. sphaericus 205y and M. tuberculosis LipQ, which have the active site Ser coded by TCT, probably have a common ancestry (FIG. 7). The lipase from subfamily I.4 such as B. subtilis, and also su Bacillus sphaericus 205y bfamily I.5 such as B. sterothermophilus, and B. thermocatenulatus have the Ser codon coded by AGC. Therefore, the lipase from Bacillus sphaericus 205y and lipases from other Bacilli were different. The lipase from Bacillus sphaericus 205y appeared to be a novel lipase as it has the pentapeptide (Gly-X-Ser-X-Gly) (SEQ ID NO:8) instead of (Ala-X-Ser-X-Gly) (SEQ ID NO:16) that was found in the Bacillus species.

Expression Under lac Promoter Regulation

The native promoter and Shine-Dalgarno region of the lipase gene were removed and fused to the pUC 19 in frame with the lac promoter. The expression system was designed to utilize the lac promoter and ribosome binding site of pUC 19. Transcription of the gene was therefore under control of the lac promoter. In this way the expression of the enzyme can be regulated with IPTG addition. The gene was amplified via PCR and after purification the resulting fusion gene plasmid (placLIP) was used to transform E. coli BL21. Transformants that showed a clear zone on tributyrin-amp agar were selected. Their plasmids were analysed by agarose gel electrophoresis for identification of insertion of the lipase gene into pUC19. The recombinant expression plasmid was regarded as placLIP.

The expression under the native promoter of Bacillus sphaericus 205y was low. Expression of lipase was significantly improved under the lac promoter regulation. When E. coli (placLIP) was cultivated in LB broth containing ampicilin and 1 mM IPTG the lipase activity reached 0.1 U ml⁻¹ min⁻¹. A ten fold increase from the lipase expression under the native promoter (0.01 U ml⁻¹ min⁻¹) was observed. However, very low activity was detected in the supernatant. This was due to the lack of effective secretory system in E. coli. About 90% of the lipase activity measured was intracellular. Protein expression was also monitored and analysed with SDS-PAGE. E. coli cells harbouring the pUC 19 (control) and placLIP were grown in LB broth containing 200 μg of ampicillin to A₆₀₀ 0.5-0.6.

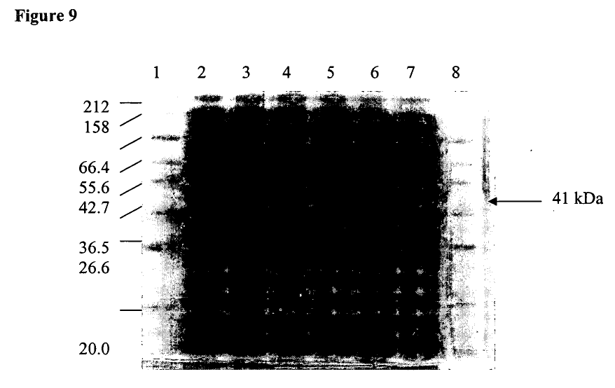

Then IPTG (1 mM) was added to the culture to induce expression. After 2 h of induction with IPTG, lipase activity detected and optimal lipase activity was obtained after 3 hour of IPTG induction. The size of the expressed lipase was about 41 000 Da correspond to the mature protein after signal peptide cleavage (FIG. 9).

Crude lipase liberated by Bacillus sphaericus 205y exhibited stability in organic solvent (Table 4). In order to determine the possibility that the cloned gene encoded the organic solvent stable lipase, the effects of similar organic solvents on the stability of the recombinant enzyme was conducted.

By contrast, n-hexane (log P 3.6) and ρ-xylene (log P 3.1) were slightly enhanced to 1.10 folds and retained 90% the lipase activity, respectively. Enzymes are often much more stable in solutions containing hydrophilic or hydrophobic organic solutions than in organic solvent-free aqueous solution. The replacement of some water molecules of an enzyme with solvent molecules sometimes stabilized the structure of the enzyme (Ogino and Ishikawa 2001).

Hexadecane, which has a high log P value of 8.8, completely inhibited the lipase activity of this recombinant enzyme. Solvent of high log P value such as hexadecane was reported to have detrimental effect on lipase activity (Basri et al. 1997). This may due to the relatively high viscosity of the solvents, which hindered efficient interaction between enzymes and substrates.

Stability studies using various organic solvents at 25% (v/v) showed that the recombinant lipase was not only stable but also slightly activated by n-hexane and therefore can be used for reaction in media containing organic solvents. The stabilities of recombinant lipase expressed in E. coli display similar trend to that secreted by Bacillus sphaericus 205y, which is stable in n-hexane and ρ-xylene.

However, in order to conclude that the cloned gene encodes the organic solvent stable lipase, the characteristics (including N-terminal sequences) of the purified recombinant lipase expressed in E. coli and that secreted by B. sphaericus 205y will be compared.

Table 4: Effect of different organic solvents on crude recombinant lipase activity. Three milliliters of cell-free supernatant of the culture was incubated with 1 ml of organic solvents at 37° C. with shaking. The remaining lipase activity relative to the non-solvent containing control was measured.

| | | Relative activity (%) at |
|---|---|---|
| Organic solvents | Log P | concentration of 25% |
| Control | — | 100 |
| Dimethyl sulfoxide | −1.22 | 10 |
| Acetonitrile | −0.15 | 0 |
| ρ-Xylene | 3.1 | 90 |
| n-Hexane | 3.6 | 110 |
| Hexadecane | 8.8 | 0 |

1. An isolated culture of Bacillus sphaericus that inhibits lipase activity while in the presence of an organic solvent.

2. The culture of claim 1, wherein said Bacillus sphaericus is an organic solvent-tolerant bacterial cell deposited with accession number DSM17161.

3. The culture of claim 1, wherein the Bacillus sphaericus strain comprises a lipase producing gene comprising all or part of SEQ ID No:4.

4. A method of identifying an organic solvent-tolerant Bacillus sphaericus 205y comprising the steps:
a) isolating said bacteria in a basal culture media.
b) screening said bacteria in liquid culture medium, wherein said medium is selected from the group consisting of M1, GYP, M3, and M5; and
c) identifying a chromosomal DNA from Bacillus sphaericus 205y comprising SEQ ID No:3.

5. The method of claim 4, wherein the basal culture media contains:
(w/v)
K₂HPO₄ 0.5%, NH₄Cl (1.0%), Na₂SO₄ (2%), KNO₃ (2%), and MgSO₄.7H₂O (0.2%).

6. The method of claim 4, wherein the basal culture medium is selected from the group consisting of benzene, toluene, ethylbenzene and ρ-xylene; and wherein hydrocarbon is added to the basal culture medium.

7. The method of claim 4, wherein the liquid culture media is selected from the following compositions: (w/v)
a) M1: peptone 3%, yeast extract 1%, NaCl 0.5%, olive oil 1%.
b) GYP: Glucose 2%, yeast extract 1%, peptone 1%, CH₃COONa.3H₂O, 1%, MgSO₄.7H₂O 0.03%, MnSO₄ 0.01%, KCl 0.05%, olive oil 2%;
c) M3 (w/v): Nutrient broth 0.325%, gum arabic 1%, CaCl₂.2H₂O 0.05%,Tween 80 1% (v/v), olive oil 1%;
d) M5: Nutrient broth 0.8% (w/v) (Oxoid),triolein 1%.

8. The method of claim 7, wherein the culture media is at pH 7.

9. A nucleotide sequence encoding a lipase, wherein said nucleotide sequence comprises all or part of SEQ ID No:4.

10. A nucleotide sequence encoding a lipase wherein said lipase comprises all or part of SEQ ID No:5.

11. The nucleotide sequence of claim 9, wherein said sequence is isolated from a Bacillus sphaericus.

12. The nucleotide sequence of claim 10, wherein said sequence is isolated from a Bacillus sphaericus.

13. A method for recombinantly producing lipase comprising the steps of:
a) providing an expression vector comprising at least one polynucleotide sequence encoding a lipase;
b) transforming a host bacterium with the expression vector;
c) growing the transformed bacterium in step (b), and
d) isolating lipase produced in step (c).

14. A method for obtaining a nucleotide sequence encoding a lipase, comprising of the steps of:
i) extracting chromosomal DNA from Bacillus sphaericus 205y;
ii) partially digestin the chromosomal DNA with restriction enzymes, wherein the restriction enzymes comprise Sau 3Al;
iii) optimizing a digested fragment between 2 kilobase to 10 kilobase; and
iv) obtaining a recombinant clone of 205y pLIP.

15. The method of claim 14, wherein the gene is derived from a mesophilic microorganism encoding Bacillus sphaericus 205y.

16. A primer pair for amplifying a nucleotide sequence encoding lipase activity, wherein said primer pair consists of SEQ ID NO:1 and SEQ ID NO:2.

17. An isolated recombinant cell comprising a nucleotide encoding SEQ ID NO:5.


SEQUENCE LISTING (27 sequences)

SEQ ID NO: 1 - DNA, 37 bp, Bacillus sphaericus
ccgaattcgtcgacaacagagtttgatcctggctcag

SEQ ID NO: 2 - DNA, 37 bp, Bacillus sphaericus
cccgggatccaagcttacggctaccttgttacgactt

SEQ ID NO: 3 - DNA, 1459 bp, Bacillus sphaericus
gccctcgagtttgatcctggctcaggacgaacgctggcggcgtccctaatacatgcaagtcgagcgaacagaaaaggagcttgctcctttgacgttagcggcggacgggtgagtaacacgtgggcaacctaccttatagtttgggataactccgggaaaccggggctaataccgaataatctatttcacttcatggtgaaatactgaaagacggtctcggctgtcgctataagatgggcccgcggcgcattagctagttggtgaggtaacggctcaccaaggcgacgatgcgtagccgacctgagagggtgatcggccacactgggactgagacacggcccagactcctacgggaggcagcagtagggaatcttccacaatgggcgaaagcctgatggagcaacgccgcgtgagtgaagaaggttttcggatcgtaaaactctgttgtaagggaagaacaagtacagtagtaactggctgtaccttgacggtaccttattagaaagccacggctaactacgtgccagcagccgcggtaatacgtaggtggcaagcgttgtccggaattattgggcgtaaagcgcgcgcaggcggtcctttaagtctgatgtgaaagcccacggctcaaccgtggagggtcattggaaactgggggacttgagtgcagaagaggaaagtggaattccaagtgtagcggtgaaatgcgtagagatttggaggaacaccagtggcgaaggcgactttctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccgttgaccactgtagagatatggttttcccttcggggacaacggtgacaggtggtgcatggttgtcgtcagctcgtgtcgtgagatgttgggttaagtcccgcaacgagcgcaacccttgatcttagttgccatcatttagttgggcactctaaggtgactgccggtgacaaaccggaggaaggtggggatgacgtcaaatcatcatgccccttatgacctgggctacacacgtgctacaatggacgatacaaacggttgccaactcgcgagagggagctaatccgataaagtcgttctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtggggtaacctt

SEQ ID NO: 4 - DNA, 1559 bp, Bacillus sphaericus
gtgaccaccccgtagctatgattaatggttgcgggtattttttgtaatattaagtgtgatttgtgcgagtagaagtaacatatggtgacaaaattgaaactactagatgaactttgggctgcatttttaaatgggaatgataataacatatcgtatgtgttaaaccgtcgaacgggtgaagttttttgaaataaaggaggtattaaaaagatgaatcagataacaaattggcgacctagaggttttcttcagtggctactcacaatcctctctgtgatcgtatgttttataatcattatagtggcatactatatttttaatccaagtaatatggataaaataatgagtctccttgcatgggggacttctatatttccgattatgctagtagttacagcattcataataatcgttttactagctctttctttttggaagaaaacattcattgctcttacagtgttatttcctattttgttgttattaatgtttttaactgtgcagccaattagtactatgaaaagctatgctaaaagcgagaatgtatcggttgctcttagctcacattttttctataaccaaaatatctcaacaaaacctagtgtagatgtggtgtatgggaaaactacggacggcattgaactaaagcttgatgtgtggccagcaaagaaaaaatcagaggatgttttaacacctgtcttgttcaagtgcatgggggtggttgggtaagtggagataaaggccaagtgcaagactggaatcagtggatgaatgatcaagggtacactgtttttgatgtccaataccgtatgcctcctgtagcaggctggaaagatgaagtgggggatgttaaatcagcgattggttggattgtgcaacatgcagatacgtataagattgatccgaacagaatcattttaatgggagagtctgctgggggaaatctagctatgcttgctgcctatagtttgggagataagcatttaccgccatcaacggatgttccggatgtaccgatcaaggcagtgattaacatgtatgggccgtcagatatgactgcgttttacaagaacaatccaagtaagcgttatgtccaagatgttctggatcagtacattggaggttcaccatccgattaccctgcgcgttataagaagctttcccctataagctatatccaggaacatacgccaccaactattatgtttttagggacgggtgatcgtattgttcctgtagagcaggcaaacgtattggatgacaaactgacgacaagcggtgtagcgcatgaactttatcttcttccaaaggtcgatcatggctttgatgcaaatccaggtagtttgagcacgcaatttgccaaggagaaagtaaaagcatttcttcaaaaatataataaataatcattatgcaatagaaaaacctttgaagacattaaccttcaaaggctttttttagcaagtacatgtagaatttattatccgtcccaaagtatatatgaaaatgcttactgcaatttggattggaaagagtatgacaatgcttacagtttttactat

SEQ ID NO: 5 - protein, 397 aa, Bacillus sphaericus
MNQITNWRPRGFLQWLLTILSVIVCFIIIIVAYYIFNPSNMDKIMSLLAWGTSIFPIMLVVTAFIIIVLLALSFWKKTFIALTVLFPILLLLMFLTVQPISTMKSYAKSENVSVALSSHFFYNQNISTKPSVDVVYGKTTDGIELKLDVWPAKKKSEDVLTPVIVQVHGGGWVSGDKGQVQDWNQWMNDQGYTVFDVQYRMPPVAGWKDEVGDVKSAIGWIVQHADTYKIDPNRIILMGESAGGNLAMLAAYSLGDKHLPPSTDVPDVPIKAVINMYGPSDMTAFYKNNPSKRYVQDVLDQYIGGSPSDYPARYKKLSPISYIQEHTPPTIMFLGTGDRIVPVEQANVLDDKLTTSGVAHELYLLPKVDHGFDANPGSLSTQFAKEKVKAFLQKYNK

SEQ ID NO: 6 - DNA, 36 bp, Bacillus sphaericus
ggcggaggtatgaattcgatgaatcagataacaaat

SEQ ID NO: 7 - DNA, 36 bp, Bacillus sphaericus
aggttaagtcttcaagttgtcgactgcataatgatt

SEQ ID NO: 8 - protein, 5 aa, Bacillus sphaericus
  MISC_FEATURE (2)..(2): Xaa may be any naturally-occurring amino acid
GXSXG

SEQ ID NO: 9 - protein, 22 aa, Bacillus sphaericus
QWLLTILSVIVCFIIIIVAYYI

SEQ ID NO: 10 - protein, 30 aa, Bacillus sphaericus
IMSLLAWGTSIFPIMLVVTAFIIIVLLALS

SEQ ID NO: 11 - protein, 27 aa, Bacillus sphaericus
FWKKTFIALTVLFPILLLLMFLTVQPI

SEQ ID NO: 12 - protein, 9 aa, Bacillus sphaericus
TPVIVQVHG

SEQ ID NO: 13 - protein, 9 aa, Bacillus sphaericus
VKSAIGWIV

SEQ ID NO: 14 - protein, 10 aa, Bacillus sphaericus
GNLAMLAAYS

SEQ ID NO: 15 - protein, 9 aa, Bacillus sphaericus
VPIKAVINM

SEQ ID NO: 16 - protein, 5 aa, Bacillus sp.
  MISC_FEATURE (2)..(2): Xaa may be any naturally-occurring amino acid
AXSXG

SEQ ID NO: 17 - protein, 108 aa, Bacillus subtilis
PVVMVHGIGGASFNFAGIKSYLVSQGWSRDKLYAVDFWDKTGTNYNNGPVLSRFVQKVLDETGAKKVDIVAHSMGGANTLYYIKNLDGGNKVANVVTVGGANRLTTGK

SEQ ID NO: 18 - protein, 108 aa, Bacillus pumilis
PVVMVHGIGGASYNFFSIKSYLATQGWDRNQLYAIDFIDKTGNNRNNGPRLSRFVKDVLDKTGAKKVDIVAHSMGGANTLYYIKNLDGGDKIENVVTIGGANGLVSSR

SEQ ID NO: 19 - protein, 114 aa, Xyxella sp.
IVLNIVRPAGVKGILPGFIFVHGGGWVLGDFQTHERLVRDLVSDSGAVAIFVNYTPSPEARYPVAINEIYAATQWVAEHGAQINVDGQRLAIVGNSVGGNMATVVAQMAKEKGT

SEQ ID NO: 20 - protein, 119 aa, Sulfolobus solfactaricus
PVRNGDIKVRVYFPNQRENLPAVVYYHGGGFVYGNLDTHDSVCRLISKLSNTIIVSVDYRLAPEHKFPTQVYDAYDVVKWLANNGGKLSIDTSKIAVAGDSAGGNLSTVVSILDRDNGE

SEQ ID NO: 21 - protein, 145 aa, Psychrobacter sp.
ANADDGDMTIRCYQSDASSHGLGFKKADTHNSDETVLLYFHGGGFCIGDVNTHHEFCHAVCEQTGWPIVSVDYRLAPEHPAPAALKDCITAYAWLAEHCHTLGALPSRIVLAGDSAGGGLSTLIAQQLSAPSQVAWSDLGSAGQK

SEQ ID NO: 22 - protein, 141 aa, Moraxella sp.
ANADGGDMTVRCYQKSTQNSERKSTDEAAMLFFHGGGFCIGDIDTHHEFCHTVCAQTGWAVVSVDYRMAPEYPAPTALKDCLAAYAWLAEHSQSLGASPSRIVLSGDSAGGCLAALVAQQVIKPIDALWQDNNQAPAADKK

SEQ ID NO: 23 - protein, 119 aa, Pseudomonas sp. B11-1
AAAAGELDARLYRPLEEDNLPLLVFFHGGGFVMGNLDTHDNLCRSLASQTEAVVVSVAYRLAPENHFPAAPLDCYAATCWLVEHAAELGVDGRRLALAGDSAGGNLALAVSRLAAQRQG

SEQ ID NO: 24 - protein, 122 aa, Bacillus halodurans
PVSDGASIPARVYQPKASGPHPIILYYHGGAFLEGYGDINTHDNIVRALAVRTGSIVVAVGYRVAPGHPFPTAIEDSYDALDWISKNAELFNGDPANVAVVGDSAGGNIATVVSLMARDREG

SEQ ID NO: 25 - protein, 126 aa, Deinococcus radiodurans
STRGLKVVLNQSYGPDTRNKLDVYAPQNAQGAPTILFIHGGSWQGGDKSGHAFVGESLARAGYVVGVMNYRLAPQNRYPSYVQDGAAALKWLRDHAGQFGGNPNNLFVSGHSAGGFNAVELVDNAR

SEQ ID NO: 26 - protein, 121 aa, M. tuberculosis (LipH)
GYDGLTDIPVRVYWPPVVRDNLPVVVYYHGGGWSLGGLDTHDPVARAHAVGAQAIVVSVDYRLAPEHPYPAGIDDSWAALRWVGENAAELGGDPSRIAVAGDSAGGNISAVMAQLARDVGG

SEQ ID NO: 27 - protein, 118 aa, M. tuberculosis (LipQ)
GPGGRENLLDIWRRPDLAPGRRAPVLIQVPGGAWTINGKRPQAYPLMSRMVELGWICVSINYSKSPRCTWPAHIVDVKRAIAWVRENIADYGGDPDFITITGGSAGAHLAALAALSAN